(19) United States
(12) Patent Application Publication
(10) Pub. No.: US 2011/0301110 A1
(43) Pub. Date: Dec. 8, 2011

(54) TRIM33 (TIF1gamma) AS A NEW DIAGNOSTIC MARKER OF CHRONIC MYELOMONOCYTIC LEUKEMIA (CMML)

(76) Inventors: Aucagne Romain, Dijon (FR); Bastie Jean Noël, Dijon (FR); Delva Laurent, Dijon (FR); Droin Nathalie, Villejuif (FR); Solary Eric, Paris (FR)

(21) Appl. No.: 13/152,130

(22) Filed: Jun. 2, 2011

Related U.S. Application Data
(60) Provisional application No. 61/351,080, filed on Jun. 3, 2010.

(51) Int. Cl. A61K 31/706; G01N 33/53; A61P 35/02; A61K 31/245; A61K 31/502; A61K 31/353; C12Q 1/68; A61K 31/166

(52) U.S. Cl. 514/43; 435/6.14; 435/7.1; 514/619; 514/535; 514/252.01; 514/456; 435/6.12

(57) ABSTRACT

A method for diagnosing a chronic myelomonocytic leukemia (CMML) in a subject includes the steps of (i) determining the level of expression of the Trim33 (tripartite motif-containing 33) gene in a biological sample from the subject, and (ii) comparing the level of expression of the Trim33 gene in the biological sample with its normal level of expression; wherein an under-expression of the Trim33 gene is associated with CMML, and to a kit for diagnosing CMML in a subject including at least one nucleic acid probe or oligonucleotide or at least one antibody, which can be used in a such a method.

### FIELD OF THE INVENTION

The present invention relates to a new identified genetic marker to diagnose chronic myelomonocytic leukemia (CMML), more particularly TRIM33 (TIF1γ) that is suspected to behave as a tumor suppressor gene in this disease.

### BACKGROUND

Hematopoiesis is maintained by a hierarchical system where hematopoietic stem cells (HSCs) give rise to multipotent progenitors, which in turn differentiate into all types of mature blood cells. Clonal stem-cell disorders in this system lead to Acute Myeloid Leukemia (AML), Myeloproliferative Neoplasms (MPNs), Myelodysplastic Syndromes (MDS) and Myelodysplastic/Myeloproliferative disorders.

Among these disorders, myelodysplastic/myeloproliferative neoplasms include four myeloid diseases grouped in 1999 by the WHO: chronic myelomonocytic leukemia (CMML), juvenile myelomonocytic leukemia (JMML), atypical chronic myeloid leukemia (aCML) and unclassified myelodysplastic/myeloproliferative syndromes (U-MDS/MPS).

Concerning CMML, its 4 defining features include an absolute monocytosis of >1×10⁹/1, the absence of Philadelphia chromosome or BCR-ABL fusion gene or a fusion gene that include PDGFRb gene (e.g. TEL-PDGFRb fusion gene), a percentage of blast cells in the bone marrow lower than 20%, and a variable degree of dyplasia in all three lineages. Myeloblasts and promonocytes comprise less than 5% of nucleated cells in peripheral blood. Roughly half of patients present with an elevated white cell count that is commonly associated with hepatomegaly and splenomegaly, the so-called myeloproliferative form of the disease. Patients lacking these features are generally considered to have the myelodysplastic form of the disease

Recent reports suggest frequent mutations in TET2, ASXL1 and RUNX1/AML1 genes (30-50% of patients), less frequent mutations in CBL, c-CBL, K-RAS or N-RAS (15-30%) and rare mutations in IDH1, IDH2, JAK2 and FLT3 (less that 10%).

### SUMMARY OF THE INVENTION

The invention relates to a method for diagnosing a Chronic MyeloMonocytic Leukemia (CMML) in a subject, which comprises the steps of:
(i) determining the level of expression of the Trim33 (tripartite motif-containing 33) gene in a biological sample from said subject, and
(ii) comparing said level of expression of the Trim33 gene in said biological sample with its normal level of expression;
wherein an under-expression of the Trim33 gene is associated with CMML.

Advantageously, the biological sample is a blood or bone marrow sample.

In a first preferred embodiment, said method comprises the step (i) of determining the level of expression of the Trim33 gene in the monocytes and/or granulocytes subset(s) of said biological sample.

Still advantageously, the normal level of expression of the Trim33 gene is the level of expression of said gene in a control sample corresponding to a biological sample of non-tumoral cells.

In a second preferred embodiment, the level of expression of the Trim33 gene is assessed by determining the level of expression of its mRNA transcript (i.e., SEQ ID NO 2 or SEQ ID NO 4) or mRNA precursors.

In a third preferred embodiment, the level of expression of the Trim33 gene is assessed by determining the level of expression of the TRIM33 protein (i.e. SEQ ID NO 1 or SEQ ID NO 3) translated from said gene.

In a forth preferred embodiment, the method of the invention comprises the steps of:
(i) determining the level of expression of the Trim33 (tripartite motif-containing 33) gene in a biological sample from said subject indirectly by determining the level of expression of the SMAD4 (mothers against decapentaplegic homolog 4) protein (SEQ ID NO 5, Accession number NP_{—}005350) in said biological sample, and
(ii) comparing said level of expression of the SMAD4 protein in said biological sample with its normal level of expression;
wherein an over-expression of the SMAD4 protein is associated with an under-expression of the Trim33 gene, which under-expression of the Trim33 gene is associated with CMML.

In a fifth preferred embodiment, The method of the invention is a method for treating a subject suffering from CMML and further comprises a step (iii) of administrating an effective amount of DNA methyltransferase inhibitor to a subject suffering from CMML and showing an under-expression of the Trim33 gene.

Advantageously, said DNA methyltransferase inhibitor is a cytosine analogue selected in the group comprising azacitidine, decitabine and zebularine.

The invention further relates to a kit for diagnosing chronic myelomonocytic leukemia (CMML) in a subject comprising at least one nucleic acid probe or oligonucleotide or at least one antibody, which can be used in a method as disclosed previously, for determining the level of expression of the Trim33 gene.

### BRIEF DESCRIPTION OF THE DRAWINGS

FIG. 1A shows mRNA analysis of Tif1γ (RQ-PCR) in total bone marrow or spleen cells of control (Ctrl) or Tif1γ^{Δ/Δ} (Δ/Δ) mice.

FIG. 1B shows Tif1γ expression in Tif1γ^{Δ/Δ} (Δ/Δ) mice. Protein extracts were prepared from bone marrow cells of control (Ctrl) or Tif1γ^{Δ/Δ} (Δ/Δ) mice for SDS-PAGE and immuno-blotting with anti-Tif1γ antibody. Equivalent loading of lanes was controlled by the use of an anti-Hsc70 antibody.

FIG. 2 shows peripheral monocytes count of control (Ctrl) and Tif1γ^{Δ/Δ} (Δ/Δ) mice.

FIG. 3 shows an immuno-blotting analysis of Smad4, in sorted myeloid cells from control (Ctrl) or Tif1γ^{Δ/Δ} (Δ/Δ) mice.

FIG. 4 shows the expression study of Tif1γ in CMML patients. mRNA was extracted from purified monocytes of healthy donors (Ctrl) or CMML patients (Subsets 1 and 2). Tif1γ measurement was carried out by RQ-PCR. Median (red bar) is expressed in relative mRNA expression units.

FIG. 5 shows that TIF1γ expression is decreased in a subset of CMML patients. Protein extracts (samples) were prepared from purified human monocytes (CD14+ cells) for SDS-PAGE and immuno-blotting with anti-TIF1γ or anti-SMAD4 antibodies. Lanes 1 to 5 correspond to samples from healthy donors. Lanes 6 to 13 correspond to samples from CMML patients. Lanes 6 to 8 correspond from monocytes (CD14+ cells) of one CMML patient before (lane 6), during (lane 7) and after treatment (lane 8) with decitabine. Equivalent loading of lanes was controlled by the use of an anti-HSC70 antibody.

FIG. 6 shows Tif1γ expression (RQ-PCR) from monocytes of two CMML patients (#1 and #2) after 3 days of culture in absence or presence of decitabine.

FIG. 7A shows Tif1γ expression (RQ-PCR) from monocytes (CD14+ cells) of one CMML patient before and after treatment with decitabine.

FIG. 7B shows that TIF1γ expression is restored in one patient treated with decitabine. Protein extracts were prepared from purified human monocytes (CD14+ cells) for SDS-PAGE and immuno-blotting with anti-TIF1γ antibody. Equivalent loading of lanes was controlled by the use of an anti-HSC70 antibody.

### DETAILED DESCRIPTION

The invention is based on the discovery by the present inventors that a Trim33 (TIF1γ) gene under-expression in 40 weeks-old mice results in symptoms similar to CMML.

This discovery leads to the identification, by the inventors, of TRIM33 (TIF1γ) gene under-expression as a CMML marker in nearly 40% of the studied patients.

Consequently, in one aspect the present invention relates to a method for diagnosing a CMML in a subject, which comprises the steps of:
(i) determining the level of expression of the Trim33 (tripartite motif-containing 33) gene in a biological sample from said subject, and
(ii) comparing said level of expression of the Trim33 gene in said biological sample with its normal level of expression;
wherein an under-expression of the Trim33 gene is associated with CMML.

In fact, the inventors have established that the under-expression of the Trim33 mRNA or of the TRIM33 protein is associated with chronic myelomonocytic leukemia (CMML). Moreover, the inventors have established on a CMML patients' panel that nearly 40% of them show a Trim33 gene under-expression.

Trim33 (Gene ID: 51592) gene, which is also known as PTC7, RFG7, TF1G, TIF1G, FLJ32925, TIFGAMMA, ECTODERMIN or TIF1GAMMA, is thought to encode for a transcriptional corepressor. However, molecules that interact with this protein have not yet been identified. The protein encoded by this gene is a member of the tripartite motif family. This motif includes three zinc-binding domains, a RING, a B-box type 1 and a B-box type 2, and a coiled-coil region. Three alternatively spliced transcript variants for this gene have been described; however, the full-length nature of one variant has not been determined.

TRIM33 alpha variant has the 1127 amino acid sequence SEQ ID NO 1 (Accession number: NP_{—}056990) and is encoded by the nucleic acid sequence SEQ ID NO 2 (Accession number: NM_{—}015906).

TRIM33 beta variant has the 1110 amino acid sequence SEQ ID NO 3 (Accession number: NP_{—}148980) and is encoded by the nucleic acid sequence SEQ ID NO 4 (Accession number: NM_{—}033020).

As used herein, the term “subject” refers to a mammal, preferably a human.

Said subject may be healthy, but the method of the invention is particularly useful for testing a subject thought to develop or to be predisposed to developing chronic myelomonocytic leukemia (CMML). In that case, the method of the invention enables to confirm that said subject develops or is predisposed for developing chronic myelomonocytic leukemia (CMML).

As used herein, the expression “biological sample” refers to solid tissues such as, for example, a bone marrow biopsy, a splenoctomy or any tissue biopsy in case of cell infiltration; and to fluids and excretions such as for example, blood, serum or plasma. Preferably, said biological sample is a blood, bone marrow sample or any tissue biopsy in case of cell infiltration, preferably a blood or bone marrow sample.

Advantageously, the method of the invention comprises the step (i) of determining the level of expression of the Trim33 gene in the monocytes and/or granulocytes subset(s) of said biological sample, preferably in the monocytes subset of said biological sample.

As used herein, the “under-expression” of the Trim33 gene occurs when the transcription and/or the translation of said gene is lower than the standard error of the assay employed to assess expression, and is preferably at least 50% inferior to the normal level of expression of said gene, preferably at least 75% inferior to the normal level of expression of said gene, and most preferably at least 85% inferior to the normal level of expression of said gene.

As used herein, the “normal level of expression” of the Trim33 gene is the level of expression of said gene in a control sample corresponding to a biological sample of non-tumoral cells, preferably in the monocytes and/or granulocytes subset(s) subset of said biological sample of non-tumoral cells, most preferably in the monocytes subset of said biological sample of non-tumoral cells. Said biological sample of non-tumoral cells can be simply obtained, as an example, from a healthy subject such as by purification of peripheral blood monocytes sorted from a healthy donor blood sample.

Said normal level of expression is assessed in a control sample and preferably is the average expression level of said gene in several control samples.

Methods for analyzing the expression of a gene are well known for the man skilled in the art.

In a particular embodiment of the invention, the level of expression of the Trim33 gene is assessed by determining the level of expression of its mRNA transcript (i.e., SEQ ID NO 2 or SEQ ID NO 4) or mRNA precursors, such as nascent RNA, of said gene.

Such analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TAQMAN), and probes arrays such as GENECHIP™ DNA Arrays (AFFYMETRIX).

Advantageously, the analysis of the expression level of mRNA transcribed from the TRIM33 gene involves the process of nucleic acid amplification, e. g., by RT-PCR (the experimental embodiment set forth in U.S. Pat. No. 4,683,202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 87, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another particular embodiment, the expression of the Trim33 gene is assessed by determining the level of expression of the TRIM33 protein (i.e. SEQ ID NO 1 or SEQ ID NO 3) translated from said gene.

Such analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from Trim33 gene. Said analysis can be assessed by a variety of techniques well known by one of skill in the art including, but not limited to, enzyme immunoassay (EIA), radioimmunoassay (RIA), Western blot analysis and enzyme linked immunoabsorbant assay (ELISA).

Polyclonal antibodies can be prepared by immunizing a suitable animal, such as mouse, rabbit or goat, with the TRIM33 protein (SEQ ID NO:1 or SEQ ID NO 3) or a fragment thereof (e.g., at least 10 or 15 amino acids). The antibody titer in the immunized animal can be monitored over time by standard techniques, such as with an ELISA using immobilized polypeptide. At an appropriate time after immunization, e.g., when the specific antibody titers are highest, antibody producing cells can be obtained from the animal and used to prepare monoclonal antibodies (mAb) by standard techniques, such as the hybridoma technique originally described by KOHLER and MILSTEIN (Nature, vol. 256, p:495-497, 1975), the human B cell hybridoma technique (KOZBOR et al., Immunol., vol. 4, p: 72, 1983), the EBV-hybridoma technique (COLE et al., In Monoclonal Antibodies and Cancer Therapy, Alan R. Liss, Inc., p: 77-96, 1985) or trioma techniques. The technology for producing hybridomas is well known (see generally Current Protocols in Immunology, COLIGAN et al. ed. , John Wiley & Sons, New York, 1994). Hybridoma cells producing the desired monoclonal antibody are detected by screening the hybridoma culture supernatants for antibodies that bind the polypeptide of interest, e.g., using a standard ELISA.

Monoclonal antibodies directed against TRIM33 are well known from the skilled person such as the antibodies commercialized by ABD SEROTEC, ABGENT, ABNOVA CORPORATION, LIFESPAN BIOSCIENCES, SANTA CRUZ BIOTECHNOLOGY Inc., or SIGMA-ALDRICH.

In still another preferred embodiment, the method of the invention comprises a step (i) of determining the level of expression of the Trim33 (tripartite motif-containing 33) gene in a biological sample from said subject indirectly by determining the level of expression of the SMAD4 (mothers against decapentaplegic homolog 4) protein (SEQ ID NO 5, Accession number NP_{—}005350) in said biological sample, and a step (ii) of comparing said level of expression of the SMAD4 protein in said biological sample with its normal level of expression;
wherein an over-expression of the SMAD4 protein is associated with an under-expression of the Trim33 gene, which under-expression of the Trim33 gene is associated with CMML.

In fact, the inventors have established that SMAD4 protein over-expression is correlated with the under-expression of the Trim33 gene.

Preferably, said SMAD4 protein over-expression corresponds to an over-expression in monocytes from said biological sample.

In still another embodiment, the method of the invention is a method for treating a subject suffering from CMML and the method of the invention further comprises a step (iii) of administrating an effective amount of DNA methyltransferase inhibitor to a subject suffering from CMML and showing an under-expression of the Trim33 gene.

In fact, the inventors have established that among the 30% of the patients suffering from CMML responding positively to a therapy with decitabine (i.e, a DNA methyltransferase inhibitor), increased expression of Trim33 gene after 3 cycles of the drug predicts a clinical response.

DNA methyltransferase inhibitors are well known from the skilled person.

In a preferred embodiment, said DNA methyltransferase inhibitor is a cytosine analogue and is preferably selected among azacitidine, decitabine and zebularine. Most preferably, said DNA methyltransferase inhibitor is azacitidine or decitabine.

Decitabine or 5-aza-2′-deoxycytidine (trade name DACOGEN) is the compound 4-amino-1-(2-deoxy-b-D-erythro-pentofuranosyl)-1,3,5-triazin-2(1 H)-one. Decitabine is indicated for the treatment of myelodysplastic syndromes (MDS) including previously treated and untreated, de novo and secondary MDS of all French-American-British subtypes (refractory anemia, refractory anemia with ringed sideroblasts, refractory anemia with excess blasts, refractory anemia with excess blasts in transformation, and chronic myelomonocytic leukemia) and Intermediate-1, Intermediate-2, and High-Risk International Prognostic Scoring System groups. Decitabine is believed to exert its antineoplastic effects after phosphorylation and direct incorporation into DNA. Decitabine inhibits DNA methyltransferase, causing hypomethylation of DNA and cellular differentiation or apoptosis. Decitabine-induced hypomethylation in neoplastic cells may restore normal function to genes that are critical for the control of cellular differentiation and proliferation. In rapidly dividing cells, the cytotoxicity of decitabine may also be attributed to the formation of covalent adducts between DNA methyltransferase and compound that has been incorporated into DNA. Non-proliferating cells are relatively insensitive to decitabine.

Azacitidine (trade name VIDAZA) is the compound 4-amino-1-[beta]-D-ribofuranosyl-s-triazin-2(1 H)-one. Azacitidine is an anti-neoplastic pyrimidine nucleoside analog used to treat several subtypes of myelodysplastic syndrome, diseases caused by abnormalities in the blood-forming cells of the bone marrow which result in underproduction of healthy blood cells. The drug exerts a cytotoxic effect on rapidly dividing cells, including cancerous cells, and may help restore normal function to genes controlling proper cellular differentiation and proliferation. Azacitidine is specifically indicated for the treatment of the following myelodysplastic syndrome subtypes: refractory anemia, refractory anemia with ringed sideroblasts (if accompanied by neutropenia or thrombocytopenia or requiring transfusions), refractory anemia with excess blasts, refractory anemia with excess blasts in transformation and chronic myelomonocytic leukemia. Azacitidine is believed to exert its antineoplastic effects by causing hypomethylation of DNA and direct cytotoxicity on abnormal haematopoietic cells in the bone marrow. The concentration of azacitidine required for maximum inhibition of DNA methylation in vitro does not cause major suppression of DNA synthesis. Hypomethylation may restore function to genes that are critical for differentiation or proliferation. The cytotoxic effects of azacitidine cause the death of rapidly dividing cells, including cancer cells that are no longer responsive to normal growth control mechanisms. Non-proliferating cells are relatively insensitive to azacitidine.

Zebularine is the compound 1-([beta]-D-ribofuranosyl)-1,2-dihydropyrimidin-2-one or 2-pyrimidone-1-[beta]-D-riboside.

In another preferred embodiment, the DNA methyltransferase inhibitor is a non-nucleoside analogue, preferably said DNA methyltransferase inhibitor is selected from procaïnamide, procaïne, hydralazine and (−)-epigallocatechin-3-gallate (EGCG).

Procaïnamide (trade names PRONESTYL, PROCAN, PROCANBID) is the compound 4-amino-[Lambda]/-(2-diethylaminoethyl)benzamide. Procaïnamide has been shown to inhibit DNA methyltransferase activity and reactivate silenced gene expression in cancer cells by reversing CpG island hypermethylation. Procaïnamide specifically inhibits the hemimethylase activity of DNA methyltransferase 1 (DNMT1), the mammalian enzyme thought to be responsible for maintaining DNA methylation patterns during replication.

Procaïne is the compound 2-(diethylamino)ethyl-4-aminobenzoate. Procaïne is a DNA-demethylating agent that is understood to inhibit DNA methyltransferases by interfering with enzyme activity.

Hydralazine (APRESOLINE) is the compound 1-hydrazinophthalazine monohydrochloride.

(−)-Epigallocatechin-S-gallate (EGCG) is a catechin analogue. EGCG is understood to inhibit DNMT activity and reactivate methylation-silenced genes in cancer cells.

In still another preferred embodiment, the DNA methyltransferase inhibitor is RG108, also known as N-phthalyl-1-tryptophan. RG 108 is a DNA methyltransferase inhibitor that is understood to inhibit DNA methyltransferases by interfering with enzyme activity. In particular, RG108 is believed to reactivate tumor suppressor gene expression (p16, SFRP1 , secreted frizzled related protein-1, and TIMP-3) in tumor cells by DNA demethylation. RG 108 also inhibits human tumor cell line (HCT116, NALM-6) proliferation and increases doubling time in culture.

A person of ordinary skill in the art can easily determine an appropriate dose of one of the DNA methyltransferase inhibitor to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the DNA methyltransferase inhibitor may be administered at a dose of from 0.1 to 30 mg/kg body weight, such as from 2 to 20 mg/kg, more preferably from 0.1 to 1 mg/kg body weight.

Said DNA methyltransferase inhibitor may be administrated by oral, rectal, vaginal, parenteral, intramuscular, intraperitoneal, intraarterial, intrathecal, intrabronchial, subcutaneous, intradermal, intravenous, nasal, buccal or sublingual routes.

The DNA methyltransferase inhibitor is typically administered subcutaneously or intravenously in accordance with a physician's direction.

By way of guidance, the recommended decitabine dose is 20 mg/m² administered by intravenous infusion for 5 days. This cycle is preferably repeated every 4-6 weeks.

Patients with advanced solid tumours typically receive a 72 h infusion of decitabine at 20-30 mg/m²/day.

By way of guidance, the recommended starting dose of azacitidine is 75 mg/m² subcutaneously or intravenously, daily for 7 days.

In a second aspect, the present invention refers to a kit for diagnosing chronic myelomonocytic leukemia (CMML) in a subject comprising at least one nucleic acid probe or oligonucleotide or at least one antibody, which can be used in a method as disclosed previously, for determining the level of expression of the Trim33 gene.

Preferably, the oligonucleotide is at least one PCR primer, preferably a set of PCR primers is provided, which allows to amplify the Trim33 gene or a fragment thereof. The skilled person readily provides such an oligonucleotide or set of PCR primers which allows to amplify a region of the Trim33 gene, provided that the nucleic acid sequence of Trim33 is well known (SEQ ID NO 2 and SEQ ID NO 4)

In a preferred embodiment, the kit comprises at least the PCR primer pair hTIF1g-S: AGCAACGGCGACATCCA (SEQ ID NO 6) and hTIF1g-AS: TGCATTCTTGGCGGCATA (SEQ ID NO 7) for determining the level of expression of the Trim33 gene.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The present kits can also include one or more reagents, buffers, hybridization media, nucleic acids, primers, nucleotides, probes, molecular weight markers, enzymes, solid supports, databases, computer programs for calculating dispensation orders and/or disposable lab equipment, such as multi-well plates, in order to readily facilitate implementation of the present methods. Enzymes that can be included in the present kits include nucleotide polymerases and the like. Solid supports can include beads and the like whereas molecular weight markers can include conjugatable markers, for example biotin and streptavidin or the like.

In one embodiment, the kit is made up of instructions for carrying out the method described herein for diagnosing a myeloid cancer in a subject. The instructions can be provided in any intelligible form through a tangible medium, such as printed on paper, computer readable media, or the like.

In the following, the invention is described in more detail with reference to amino acid sequences, nucleic acid sequences and the examples. Yet, no limitation of the invention is intended by the details of the examples. Rather, the invention pertains to any embodiment which comprises details which are not explicitly mentioned in the examples herein, but which the skilled person finds without undue effort.

### EXAMPLES

### 1) Tif1γ^{Δ/Δ} Mice Develop a Myeloproliferative Disease with Monocytic Features

To get further insights in the contribution of TIF1γ to hematopoiesis, we generated mice selectively deficient for Tif1γ, by breeding floxed Tif1γ mice (Tif1γ^{f/f}; DOISNE et al., J. Exp. Med., vol. 206(6), p:1365-1378, 2009) with cFES-Cre transgenic animals. In litters of such crosses, the cFES-Cre; Tif1γ^{f/f} (Tif1γ^{Δ/Δ} mice correspond to the hematopoietic tissues-restricted knock out mice whereas the Tif1γ^{f/f} mice represent controls. While the Tif1γ null mice invariably died at perinatal time points, Tif1γ^{Δ/Δ} mice reached adulthood and were fertile.

For RQ-PCR analysis, total RNA was isolated from BM cells and splenocytes using TRIZOLreagent (INVITROGEN) according to the manufacturer's instructions. cDNA was obtained from 150 ng of total RNA using M-MLV Reverse Transcriptase (PROMEGA). RQ-PCR was performed in triplicates using TAQMAN® probes (APPLIED BIOSYSTEMS) and analyzed in an APPLIED BIOSYSTEMS 7500 Real-Time PCR System. The TAQMAN® assay was the following: Tif1γ (Mm01308706_m1). Values for each PCR were normalized with Hprt (Mm03024075_m1).

For western blotting analysis, the proteins extracted from mouse bone marrow cells were denaturated by boiling in LAEMMLI buffer then separated by SDS-PAGE and electro-blotted to nitrocellulose membranes. Membranes were blocked in 1×PBS-T (0.1%) and fat-free dry milk (5%) (blocking buffer) during 1 h at room temperature. Membranes were incubated with the primary antibody (anti-Tif1γ (SANTA CRUZ BIOTECHNOLOGY)) diluted in the blocking buffer at 4° C. overnight. Then, the membranes were washed three times in 1×PBS-T (0.1%) during 10 min each. Secondary antibodies conjugated with horseradish peroxidase were added, and the membranes were incubated at room temperature during 1 h. Membranes were then washed three times in 1×PBS-T (0.1%) during 10 min each. ECL Western blotting reagent kit (MILLIPORE) was used for protein detection. Equivalent loading of lanes was controlled by PONCEAU Red Stain and the use of an anti-Hsc70 antibody (SANTA CRUZ BIOTECHNOLOGY).

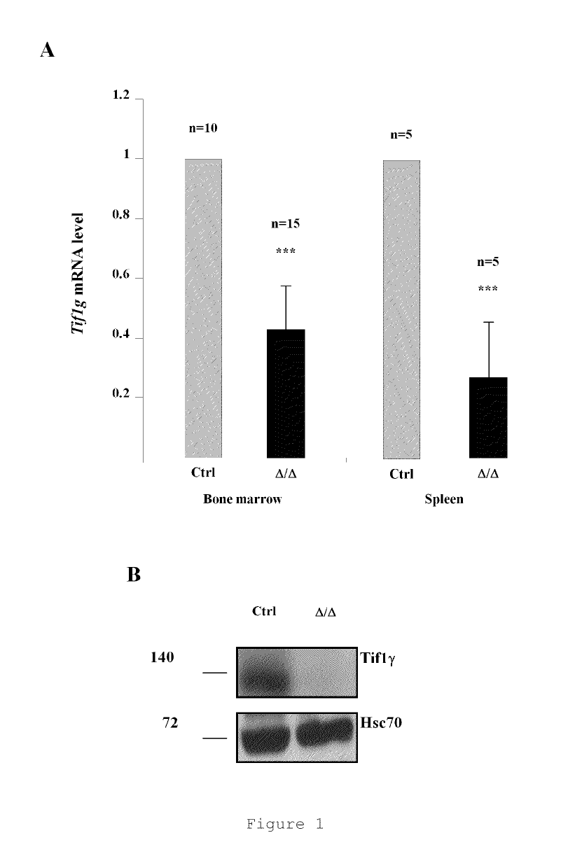

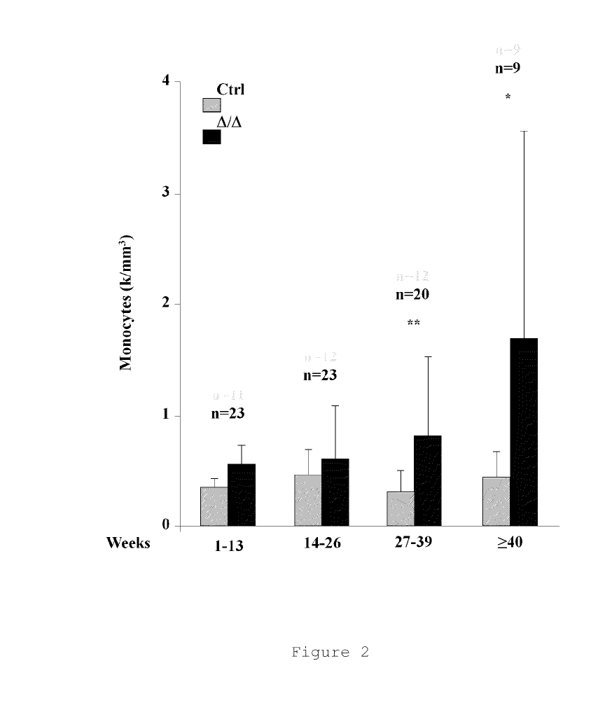

We demonstrated that the Tif1γ deletion being partial, it was associated with a low expression of Tif1γ at the RNA and protein levels in the bone marrow and the spleen (FIGS. 1A and 1B).

Mice older than 40 weeks exhibited a hyperleukocytosis (not shown) due to a monocytosis (FIG. 2). Further immunophenotypic studies indicated an accumulation with time of Gr1^{low}Mac1⁺ cells and a macroscopic analysis showed that Tif1γ^{Δ/Δ} mice displayed a hepatosplenomegaly (not shown). Histological analysis of the bone marrow showed hyper-cellularity due to monocytic hyperplasia and flow cytometry analyses confirmed an increased number of Gr1^{low}Mac1⁺ cells in the bone marrow. The results also shown that while the number of Gr1^{high}Mac1⁺ cells (granulocytes) remained unchanged, and that the number of Ter119⁺CD71⁺ cells (erythroid progenitors) was diminished in the bone marrow.

Likewise, histopathology examination demonstrated a destruction of splenic organization resulting from infiltration of the red pulp by mature myeloid cells suggesting maturing monocytes in which the expression of Tif1γ was decreased. The results demonstrated that these cells expressed Mac1 and were highly proliferative, as suggested by Ki67 staining In all cases, spleen sections showed extramedullary hematopoiesis since myeloid cells, megakaryocytes, and erythroid precursors were present. Liver was also found very often infiltrated by high proliferative cells with maturing myeloid forms.

### 2) Effect of Tif1γ on HSCs and Hematopoietic Progenitor Populations in the Bone Marrow

To dissect the effect of Tif1γ deletion on progenitor populations, the bone marrow cells of Tif1γ^{Δ/Δ} mice were analyzed immunophenotypically for the percentage of LSK (Lin⁻Sca-1⁺c-Kit⁺), CMP (Lin⁻Sca-1⁻c-Kit⁺CD34⁺CD16/32⁻), GMP (Lin⁻Sca-1⁻c-Kit⁺CD34⁺CD16/32⁺) and MEP (Lin⁻Sca-1⁻IL7Rα⁻c-Kit⁺CD34⁻CD16/32⁻) populations.

The results showed that the number of CMPs was dramatically reduced (i.e. 3-fold) whereas the number of GMPs increased by approximately 5-fold compared with the respective control compartments. In contrast, the number of MEPs decreased strikingly in Tif1γ^{Δ/Δ} deficient mice (i.e. 3-fold).

Thus, T1γ deletion leads to a selective expansion of the GMPs and a suppression of the MEPs, associated with a deficiency of the development from very primitive progenitors (i.e., SLAM population) to CMPs.

The pool size of LSK cells in the bone marrow was significant increased (i.e. 2-fold) in young Tif1γ^{Δ/Δ} mice compared with the control littermates. However, this increase was not observed in mice older than 10 months developing a monocytosis.

The number of the primitive LSK based on the “SLAM code” (CD150⁺CD48⁻) is decreased in Tif1γ^{Δ/Δ} mice compared with the controls (i.e. 2-fold). The increased number of LSK and the decreased number of LSK-SLAM cells indicates that another subpopulation of the LSK compartment is altered, which was observed to be an expansion of the MPP (LSK CD34⁺) compartment.

Finally, the loss of Tif1γ in the bone marrow alters the distribution of primitive progenitors, leading to an increase of myeloid progenitors and a decrease of erythroid progenitors. These results define the stages of the involvement of Tif1γ in hematopoiesis and implicate an important role for Tif1γ in controlling hematopoietic progenitor differentiation.

### 3) The Tif1γ^{Δ/Δ} Myeloproliferative Disease is Transplantable

Mice transplanted with Tif1γ^{Δ/Δ} bone marrow cells from 2 months old mice, survived from lethal irradiation. Two months after transplantation, cells in which Tif1γ gene was deleted were detected by Q-PCR in the blood of these mice (not shown), this deletion being correlated to the decreased expression of Tif1γ (not shown). This result indicates that the deletion occurred in HSCs. The Tif1γ^{Δ/Δ} myeloproliferative disease was transplantable into secondary recipients as determined by spleen weight, histopathologic examination and flow cytometric studies performed on lethally irradiated wild-type recipient animals, supporting the notion that the phenotypic effects of the Tif1γ deletion were cell autonomous. Indeed, the mice exhibited a monocytosis. Immunophenotypic analyses also demonstrated an increased number of Gr1^{low}Mac1⁺ cells in the bone marrow and in the spleen. The number of Gr1^{high}Mac1⁺ cells (granulocytes) remained unchanged. We confirmed that Tif1γ deletion leads to an increase of GMPs associated with a decrease of CMPs and MEPs. In addition, we observed an increase of the LSK fraction in Tif1γ^{Δ/Δ} mice compared with the control littermates. A second transplantation into lethally irradiated recipients reproduced the disease.

Altogether, these results indicate on the one hand that the repopulating capabilities of Tif1γ deficient cells were not altered, and on the other hand, that the disease due to the Tif1γ deletion was initiated from the HSCs compartment. The myeloproliferative phenotype associated with Tif1γ deletion in mice suggests that Tif1γ deficiency may contribute to transformation by a loss of proliferative control at the stem/progenitor stage or in a committed myeloid lineage.

Hence, our data identify Tif1γ as a tumor suppressor gene in mice. Tif1γ^{Δ/Δ} mice provide a suitable animal model for better understanding the progression of a premalignant disorder.

### 4) Alteration of the TGF-β/BMP Signaling Pathway in Tif1γ^{Δ/Δ} Mice

Several reports have described TIF1γ as a member of the TGF-β/BMP signaling pathway. However, its mechanism of action is still unclear. Members of the TGF-β superfamily which are involved in nearly all aspects of cell biology act through a complex system of Smads and receptor-interacting proteins.

It was hypothesized that TIF1γ may selectively bind receptor-phosphorylated Smad2/3 in competition with Smad4, and control hematopoietic cell fate through the formation of this protein complex. TIF1γ was also demonstrated to promote the development of ectoderm at the expense of mesoderm in Xenopus eggs by antagonizing the TGF-β superfamily signaling. In mammalian tissues and cell lines, TIF1γ was identified as a general inhibitor of Smad-dependent TGF-β signaling, which was first explained by TIF1γ-mediated Smad4 ubiquitination and proteasome-mediated degradation. Recently, TIF1γ was suggested instead to serve as a Smad4 monoubiquitin ligase that regulates Smad4 functions rather than degradation.

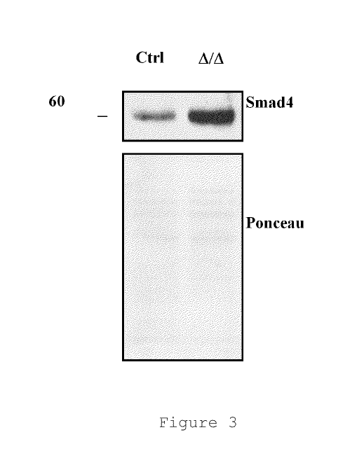

To further explore the link between TIF1γ and Smad4, we examined Smad4 expression in the bone marrow of Tif1γ^{Δ/Δ} mice. Tif1γ gene deletion did not affect Smad4 mRNA level (data not shown). Interestingly, we identified a very high level of Smad4 protein in sorted myeloid cells of Tif1γ^{Δ/Δ} mice (FIG. 3). In addition, in spleen of Tif1γ^{Δ/Δ} mice, we observed a very high number of positive cells for Smad4 with a cytoplasmic localization, likely corresponding to the infiltrated hematopoietic cells (data not shown).

In order to investigate more deeply the TGF-β/BMP signaling pathway, we examined the mRNA expression of its members in murine sorted GMP and MPP cells.

The Table I shows changes in gene transcription of GMPs (A) or MPPs (B) from control or Tif1γ^{Δ/Δ} mice, measured by RQ-PCR by using TAQMAN® Express Plates.

TABLE I

| Symbol | Fold change |
|---|---|
| Tdgf1 | 36.807 |
| Col1a1 | 21.007 |
| Nog | 12.972 |
| Col1a2 | 8.451 |
| Tdgfb1i1 | 2.711 |
| Ltbp4 | 2.577 |
| Ltbp1 | 2.572 |
| Pdgfb | 2.541 |
| Evi1 | 2.400 |
| Tgfbr3 | 2.260 |
| Tsc22d1 | 1.858 |
| Acvr2a | 1.739 |
| Inha | 1.524 |
| Smad1 | 1.428 |
| Gdf1 Lass1 | 1.406 |
| Amh | 1.317 |
| Acvrl1 | 1.310 |
| Hoxa9 | 1.291 |
| Gata1 | 1.287 |
| Jun | 1.186 |
| Acvr1 | 1.146 |
| Tgfbr2 | 1.123 |
| Smad3 | 1.113 |
| Smurf1 | 1.074 |
| Eng | 1.068 |
| Smad2 | 1.051 |
| Cdc25a | 1.050 |
| Bmpr1a | 1.048 |
| Tgfbr1 | 1.035 |
| Il6 | 1.031 |
| Sox4 | 1.017 |
| Smad4 | 0.965 |
| Bmpr2 | 0.943 |
| Itgb5 | 0.935 |
| Bmp1 | 0.912 |
| Smad5 | 0.875 |
| Tdgfb1 | 0.875 |
| Cdkn1a | 0.857 |
| Bambi | 0.839 |
| Cebpa | 0.817 |
| Fkbp1b | 0.812 |
| Runx1 | 0.789 |
| Junb | 0.785 |
| Myc | 0.777 |
| Fos | 0.759 |
| Inhba | 0.735 |
| Id2 | 0.689 |
| Cebpb | 0.648 |
| Sfpi | 0.643 |
| Itgb7 | 0.623 |
| Plau | 0.609 |
| Gdf3 | 0.600 |
| Tgfb2 | 0.581 |
| Id1 | 0.573 |
| Stat1 | 0.566 |
| Serpine1 | 0.474 |
| Tgfbi | 0.429 |
| Igf1 | 0.371 |
| Fst | 0.033 |
| Cd79a | 0.028 |
| Smurf1 | 18.615 |
| Fos | 7.257 |
| Jun | 2.881 |
| Sox4 | 2.802 |
| Runx1 | 1.964 |
| Junb | 1.489 |
| Sfpi | 1.112 |
| Eng | 0.889 |
| Smad4 | 0.885 |
| Myc | 0.785 |
| Smad2 | 0.709 |
| Tdgfb1 | 0.653 |
| Cebpa | 0.565 |
| Smad5 | 0.529 |
| Smad1 | 0.489 |
| Bmpr2 | 0.317 |
| Itgb7 | 0.298 |
| Cebpb | 0.259 |
| Col1a2 | 0.026 |
| Amh | 0.002 |
| Smurf1 | 18.615 |

We identified modified expression levels of several members of this pathway in Tif1γ^{Δ/Δ} mice (Table I). Interestingly, the strongest variation observed in MPP cells corresponds to the overexpression of Smurf1. Smurf1 was originally identified as an E3 ubiquitin ligase that interacts with Smad1 and induces its degradation. In the current model, Smads mediate the signal from the membrane into the nucleus. Phosphorylation of Smad1 leads to its interaction with Smad4. Then, this complex translocates from the cytoplasm to the nucleus. It accumulates in the nucleus where it is implicated in transcriptional regulation by sequestering transcription factors or by interacting with promoters of its target genes.

In Tif1γ^{Δ/Δ} mice, the low level of Smad1 at the transcriptional level (Table I) may lead to the lack of Smad1-Smad4 heterodimers formation and translocation, and a subsequent cytoplasmic accumulation of Smad4, preventing its activity.

Furthermore, Smurf1 interacts with Smad7 also inducing its ubiquitin-dependent degradation. Interestingly, Smad7 functions as an adaptor for WWP1 and Nedd4-2, two ubiquitin ligases, in the ubiquitination and degradation of Smad4. Thus, the overexpression of Smurf1 in mice may induce the degradation of Smad7, preventing the ubiquitination and degradation of Smad4. Altogether, the lack of Tif1γ associated to the overexpression of Smurf1 could lead to a higher expression of an inactive form of the protein Smad4.

We asked whether the deregulation of TGF-β genes belonging to the eponymous signaling pathway could affect the hematopoietic stem cell fate, by impairing the responsiveness of Tif1γ-deficient HSCs to TGF-β stimulation, contributing thus to the increased myelopoiesis observed in Tif1γ-deficient mice.

To answer this question, control and Tif1γ-deficient Lin⁻ cells were cultured for 10 days in the presence or absence of TGF-β. Cells were also treated with TGF-β inhibitor. As expected, stimulation with TGF-β limited formation of myeloid cells from control cells compared to untreated cultures. In contrast, similar treatments did not inhibit myelopoiesis of Tif1γ-deficient Lin⁻ cells. Similarly, pharmacological inhibition of TGF-β signalling by the TGF-β inhibitor in control Lin⁻ cells restored partially the myeloid differentiation.

These results suggest that the lack of response to TGF-β contributes to the ability of Tif1γ-deficient HSCs to constitutively overproduce myeloid progenitors and to induce CMML development in vivo.

### 5) Low Expression of TIF1γ in One Subset of Chronic Myelomonocytic Leukemia Patients

In view of the results obtained in Tif1γ-deficient mouse, we hypothesized that TIF1γ expression may be down-regulated in human CMML cells.

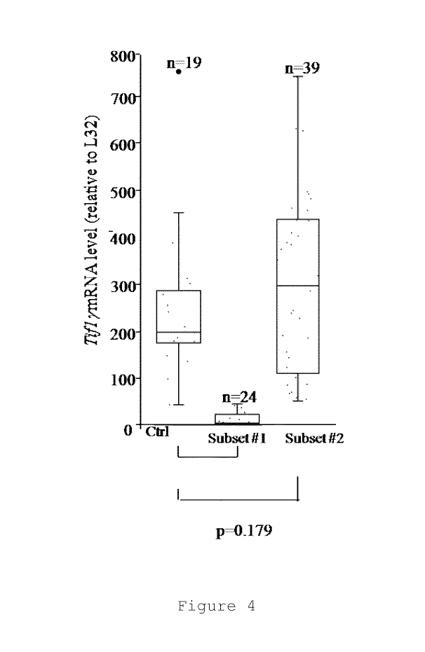

To test this hypothesis, we examined first by RQ-PCR the expression level of TIF1γ in monocytes obtained from 63 CMML patients compared to 19 healthy donors. RQ-PCR analysis demonstrated that TIF1γ gene expression was almost undetectable in 24 out of the 63 patients (38%, FIG. 4). Next, by immunohistochemistry we examined TIF1γ pattern in human hematopoietic cells. We measured lower level of TIF1γ in monocytes as well as in granulocytes, compared to lymphocytes from the patient and control cells from a healthy donor. Finally, by immunoblotting we confirmed the decrease of TIF1γ expression in a subset of CMML patients (FIG. 5).

For Western blotting, the proteins extracted from human monocytes (CMML patients or healthy volunteers) were denaturated by boiling IN LAEMMLI buffer then separated by SDS-PAGE and electro-blotted to nitrocellulose membranes. Membranes were blocked in 1×PBS-T (0.1%) and fat-free dry milk (5%) (blocking buffer) during 1 h at room temperature. Membranes were incubated with the primary antibody (SMAD4, SANTA CRUZ BIOTECHNOLOGY) diluted in the blocking buffer at 4° C. overnight. Then, the membranes were washed three times in 1×PBS-T (0.1%) during 10 min each. Secondary antibodies conjugated with horseradish peroxidase were added, and the membranes were incubated at room temperature during 1 h. Membranes were then washed three times in 1×PBS-T (0.1%) during 10 min each. ECL Western blotting reagent kit (MILLIPORE) was used for protein detection. Equivalent loading of lanes was controlled by PONCEAU Red Stain and the use of an anti-HSC70 antibody (SANTA CRUZ BIOTECHNOLOGY).

We also observed the very high level of SMAD4 studied by immunoblot using a commercially available antibody (Anticorps monoclonal de lapin, ABCAM) in sorted monocytes from CMML patients harboring very low levels of TIF1γ (FIG. 5). As observed in Tif1γ^{Δ/Δ} mice, a high number of SMAD4 positive cells was also identified by immunohistochemistry using the same antibody in the spleen of a CMML patient harboring a very low level of Tif1γ (FIG. 4).

Thus, our data highlight a role of TIF1γ as a tumor suppressor gene in hematopoietic cells in which the inhibition of its expression may favor the appearance of CMML. The low expression of TIF1γ in one subset of CMML patients represents one of the most frequently observed anomaly (38%) and is probably the most frequent in a high risk subset of patients (50%).

### 6) Low Expression of TIF1γ in One Subset of Chronic Myelomonocytic Leukemia Patients

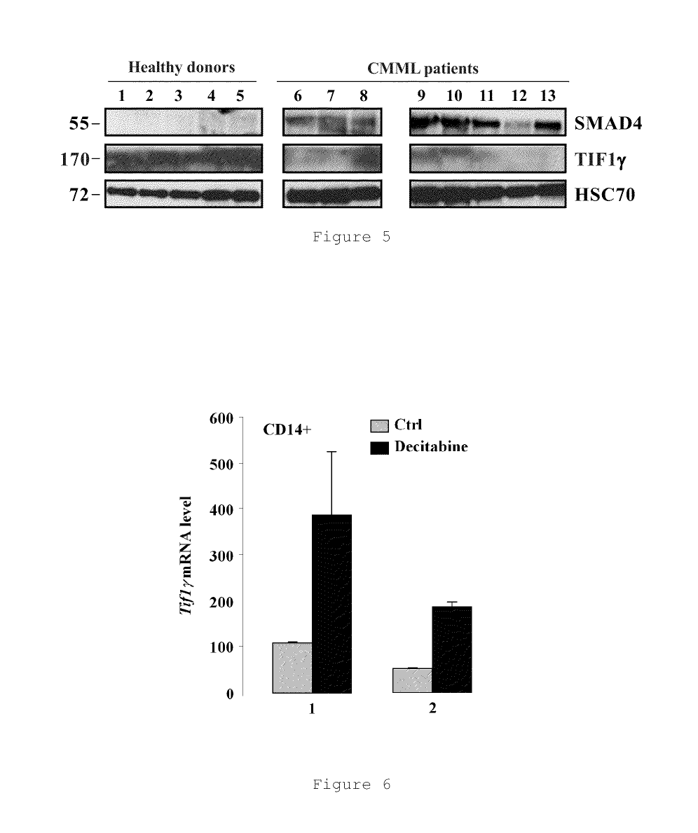

A three days exposure of PB monocytes from CMML patients to decitabine, a demethylating agent that was recently shown as a potentially efficient therapeutic in about 30% of these patients, increased the expression of Tif1γ (FIG. 6).

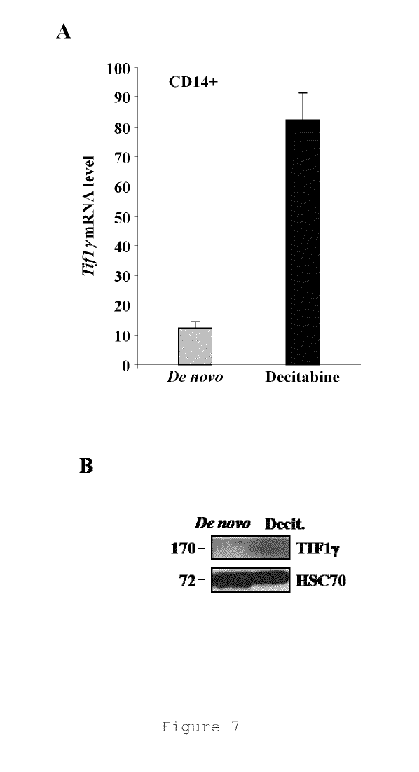

Interestingly, a CMML patient expressing a very low Tif1γ level and who achieved a complete remission after 5 cycles of decitabine, harbored a normal TIF1γ expression in his sorted PB monocytes (8-fold increase compared to the level measured before treatment) (FIGS. 7A and 7B).

Under-expression of Tif1γ gene could be a biomarker of the high-risk forms of the disease and its evolution upon treatment with demethylating agents and other epigenetic drugs could possibly predict the treatment outcome.

We claim:

1. A method for diagnosing a chronic myelomonocytic leukemia (CMML) in a subject, which comprises the steps of:
(i) determining the level of expression of the Trim33 (tripartite motif-containing 33) gene in a biological sample from said subject, and (ii) comparing said level of expression of the Trim33 gene in said biological sample with its normal level of expression;
wherein an under-expression of the Trim33 gene is associated with CMML.

2. The method of claim 1, wherein said subject is a human.

3. The method of claim 1, wherein said biological sample is a blood or bone marrow sample.

4. The method of claim 1, wherein said method comprises the step (i) of determining the level of expression of the Trim33 gene in the monocyte and/or granulocyte subsets of said biological sample.

5. The method of claim 1, wherein said under-expression of the Trim33 gene corresponds to a transcription and/or the translation of said gene that is at least 50% inferior to the normal level of expression of said gene.

6. The method of claim 1, wherein said under-expression of the Trim33 gene corresponds to a transcription and/or the translation of said gene that is at least 75% inferior to the normal level of expression of said gene.

7. The method of claim 1, wherein said under-expression of the Trim33 gene corresponds to a transcription and/or the translation of said gene that is at least 85% inferior to the normal level of expression of said gene.

8. The method of claim 1, wherein the normal level of expression of the Trim33 gene is the level of expression of said gene in a control sample corresponding to a biological sample of non-tumoral cells.

9. The method of claim 1, wherein the normal level of expression of the Trim33 gene is the level of expression of said gene in the monocyte and/or granulocyte subset of a biological sample of non-tumoral cells.

10. The method of claim 1, wherein the level of expression of the Trim33 gene is assessed by determining the level of expression of its mRNA transcript (i.e., SEQ ID NO 2 or SEQ ID NO 4) or mRNA precursors.

11. The method of claim 1, wherein the level of expression of the Trim33 gene is assessed by determining the level of expression of the TRIM33 protein (i.e. SEQ ID NO 1 or SEQ ID NO 3) translated from said gene.

12. The method of claim 1, wherein said method comprises the steps of:
(i) determining the level of expression of the Trim33 (tripartite motif-containing 33) gene in a biological sample from said subject indirectly by determining the level of expression of the SMAD4 (mothers against decapentaplegic homolog 4) protein (SEQ ID NO 5, Accession number NP_{—}005350) in said biological sample, and
(ii) of comparing said level of expression of the SMAD4 protein in said biological sample with its normal level of expression;
wherein an over-expression of the SMAD4 protein is associated with an under-expression of the Trim33 gene, which under-expression of the Trim33 gene is associated with CMML.

13. The method of claim 12, wherein said SMAD4 protein over-expression corresponds to an over-expression in monocytes from said biological sample.

14. The method of claim 1, wherein said method is a method for treating a subject suffering from CMML and the method of the invention further comprises a step (iii) of administrating an effective amount of DNA methyltransferase inhibitor to a subject suffering from CMML and showing an under-expression of the Trim33 gene.

15. The method of claim 14, wherein said DNA methyltransferase inhibitor is administered at a dose of from 0.1 to 30 mg/kg body weight.

16. The method of claim 14, wherein said DNA methyltransferase inhibitor is administered at a dose of from 2 to 20 mg/kg body weight.

17. The method of claim 14, wherein said DNA methyltransferase inhibitor is administered at a dose of from 0.1 to 1 mg/kg body weight.

18. The method of claim 14, wherein said DNA methyltransferase inhibitor is a cytosine analogue selected in the group comprising azacitidine, decitabine and zebularine.

19. The method of claim 14, wherein said DNA methyltransferase inhibitor is azacitidine or decitabine.

20. The method of claim 14, wherein said DNA methyltransferase inhibitor is decitabine.

21. The method of claim 20, wherein said decitabine is administrated at a dose of 20 mg/m² subject by intravenous infusion for 5 days.

22. The method of claim 14, wherein said DNA methyltransferase inhibitor is azacitidine.

23. The method of claim 22, wherein said azacitidine is administrated daily subcutaneously or intravenously at a dose of 75 mg/m² subject for 7 days.

24. The method of claim 14, wherein said DNA methyltransferase inhibitor is a non-nucleoside analogue selected in the group comprising procainamide, procaine, hydralazine and (−)-epigallocatechin-3-gallate (EGCG).

25. A kit for diagnosing chronic myelomonocytic leukemia (CMML) in a subject comprising at least one nucleic acid probe or oligonucleotide or at least one antibody, which can be used in a method as disclosed previously, for determining the level of expression of the Trim33 gene.

26. The kit of claim 25, wherein said kit comprises at least the PCR primer pair hTIF1g-S: AGCAACGGCGACATCCA (SEQ ID NO 6) and hTIF1g-AS: TGCATTCTTGGCGGCATA (SEQ ID No 7) for determining the level of expression of the Trim33 gene.


SEQUENCE LISTING (7 sequences)

SEQ ID NO: 1 - protein, 1127 aa, Homo sapiens
MAENKGGGEAESGGGGSGSAPVTAGAAGPAAQEAEPPLTAVLVEEEEEEGGRAGAEGGAAGPDDGGVAAASSGSAQAASSPAASVGTGVAGGAVSTPAPAPASAPAPGPSAGPPPGPPASLLDTCAVCQQSLQSRREAEPKLLPCLHSFCLRCLPEPERQLSVPIPGGSNGDIQQVGVIRCPVCRQECRQIDLVDNYFVKDTSEAPSSSDEKSEQVCTSCEDNASAVGFCVECGEWLCKTCIEAHQRVKFTKDHLIRKKEDVSESVGASGQRPVFCPVHKQEQLKLFCETCDRLTCRDCQLLEHKEHRYQFLEEAFQNQKGAIENLLAKLLEKKNYVHFAATQVQNRIKEVNETNKRVEQEIKVAIFTLINEINKKGKSLLQQLENVTKERQMKLLQQQNDITGLSRQVKHVMNFTNWAIASGSSTALLYSKRLITFQLRHILKARCDPVPAANGAIRFHCDPTFWAKNVVNLGNLVIESKPAPGYTPNVVVGQVPPGTNHISKTPGQINLAQLRLQHMQQQVYAQKHQQLQQMRMQQPPAPVPTTTTTTQQHPRQAAPQMLQQQPPRLISVQTMQRGNMNCGAFQAHQMRLAQNAARIPGIPRHSGPQYSMMQPHLQRQHSNPGHAGPFPVVSVHNTTINPTSPTTATMANANRGPTSPSVTAIELIPSVTNPENLPSLPDIPPIQLEDAGSSSLDNLLSRYISGSHLPPQPTSTMNPSPGPSALSPGSSGLSNSHTPVRPPSTSSTGSRGSCGSSGRTAEKTSLSFKSDQVKVKQEPGTEDEICSFSGGVKQEKTEDGRRSACMLSSPESSLTPPLSTNLHLESELDALASLENHVKIEPADMNESCKQSGLSSLVNGKSPIRSLMHRSARIGGDGNNKDDDPNEDWCAVCQNGGDLLCCEKCPKVFHLTCHVPTLLSFPSGDWICTFCRDIGKPEVEYDCDNLQHSKKGKTAQGLSPVDQRKCERLLLYLYCHELSIEFQEPVPASIPNYYKIIKKPMDLSTVKKKLQKKHSQHYQIPDDFVADVRLIFKNCERFNEMMKVVQVYADTQEINLKADSEVAQAGKAVALYFEDKLTEIYSDRTFAPLPEFEQEEDDGEVTEDSDEDFIQPRRKRLKSDERPVHIK

SEQ ID NO: 2 - DNA, 8339 bp, Homo sapiens
ctgcggctggggctgggggcggcggcggcggcgacgcgggcggcgggcggcgcggggcggtccggcgggttcaaagaggaaaacatggcggaaaacaaaggcggcggcgaggctgagagcggcggcgggggcagcggcagcgcgccggtaactgccggggccgccgggcccgccgcgcaggaggcggagccgcctctcaccgcggtgctggtggaggaggaggaggaggaaggcggcagggccggcgctgagggcggcgcggccgggcccgacgacgggggggtggccgcggcctcctcgggctcggcccaggctgcttcatctcctgcggcctcagtgggcactggagttgccgggggcgcagtatcgacgccggctccagctccagcctcggctcccgctccgggtccctcggcagggccgcctcctggaccgccagcctcgctcctggacacctgcgccgtgtgtcagcagagcttgcagagccggcgtgaggcggagcccaagctgctgccctgtcttcactccttctgcctgcgctgcctgcccgagccggagcgccagctcagcgtgcccatcccggggggcagcaacggcgacatccagcaagttggtgtaatacggtgcccagtatgccgccaagaatgcagacagatagaccttgtggataattattttgtgaaagacacatctgaagctcctagcagttctgatgaaaaatcagaacaggtatgtactagttgtgaagacaatgcaagtgcagttggcttttgtgtagaatgtggagagtggctatgtaagacatgtatcgaagcacatcaaagagtaaaatttactaaagatcacttgatcaggaagaaagaagatgtctcagagtctgttggagcatctggtcaacgccctgttttctgccctgtacacaaacaagaacagttgaaacttttctgtgaaacatgtgatagattgacatgtagagactgtcagctattggaacacaaagaacataggtatcagtttttggaagaagcttttcaaaatcagaagggtgcaattgagaatctactggcgaaacttcttgagaagaagaattatgttcattttgcagctactcaggtgcagaataggataaaagaagtaaatgagactaacaaacgagtagaacaggaaattaaagtggccattttcacccttatcaatgaaattaataagaaaggaaaatctctcttacaacagctagagaatgttacaaaggaaagacagatgaagttactacagcagcagaatgacatcacaggcctttcccggcaggtgaagcatgttatgaacttcacaaattgggcaattgcaagtggcagcagcacagcactactatacagcaagcgactgattactttccagttgcgtcatattttgaaagcacggtgtgatcctgtccctgctgctaatggagcaatacgtttccattgtgatcccaccttctgggcaaagaatgtagtcaatttaggtaatctagtaatagagagtaaaccagctcctggttatactcctaatgttgtagttgggcaagttcctccagggacaaaccacattagtaaaacccctggacagattaacttagcacagcttcgactccagcacatgcaacaacaagtatatgcacagaaacatcagcagttgcaacagatgaggatgcagcaaccaccagcacctgtaccaactacaacaacaacaacacaacagcatcctagacaagcagcccctcagatgttacaacaacagcctcctcgattgatcagtgtgcaaacaatgcaaagaggcaacatgaactgtggagcttttcaagcccatcagatgagactggctcagaatgctgccagaataccagggatacccaggcacagcggccctcaatattccatgatgcagccacacctccaaagacaacactcaaacccagggcatgctggaccctttcccgtagtatcggtacacaacaccacaatcaacccaacgagccctactacagcaactatggcaaatgcaaaccgaggtcccaccagcccatctgttacagcaatagagctaatcccctcagttaccaatccagaaaaccttccatcgctgccagatattccacccatacagttggaagatgctggctcaagtagtttagataatctactaagtagatacatctcaggcagtcacctacccccacagcctacaagcaccatgaatccttctccaggtccctctgccctttctccgggatcatcaggtttatccaattctcacacacctgtgagacccccaagtacttctagtactggcagtcgaggcagctgtgggtcatcaggaagaactgctgagaagacaagtcttagtttcaaatctgatcaggtgaaggtcaagcaagaacctgggactgaagatgaaatatgtagcttttcaggaggtgtaaaacaagaaaaaacagaggatggcaggaggagtgcttgcatgttgagcagtcctgagagtagcttgacaccacctctctcaaccaacctgcatctagaaagtgaattggatgcattggcaagcctggaaaaccatgtgaaaattgaacctgcagatatgaatgaaagctgcaaacagtcagggctcagcagccttgttaatggaaagtccccaattcgaagcctcatgcacaggtcggcaaggattggaggagatggcaacaataaagatgatgacccaaatgaagactggtgtgctgtctgccaaaacggaggagatctcttgtgctgcgaaaaatgtccaaaggtctttcatctaacttgtcatgttccaacactacttagctttccaagtggggactggatatgcacattttgtagagatattggaaagccagaagttgaatatgattgtgataatttgcaacatagtaagaaggggaaaactgcgcaggggttaagccccgtggaccaaaggaaatgtgaacgtcttctgctttacctctattgccatgaattaagtattgaattccaggagcctgttcctgcttcgataccaaactactataaaattataaagaaaccaatggatttatccaccgtgaaaaagaagcttcagaaaaaacattcccaacactaccaaatcccggatgactttgtggccgatgtccgtttgatcttcaagaactgtgaaaggtttaatgaaatgatgaaagttgttcaagtttatgcagacacacaagagattaatttgaaggctgattcagaagtagctcaggcagggaaagcagttgcattgtactttgaagataaactcacagagatctactcagacaggaccttcgcacctttgccagagtttgagcaggaagaggatgatggtgaggtaactgaggactctgatgaagactttatacagccccgcagaaaacgcctaaagtcagatgagagaccagtacatataaagtaaaatgacatggatttaaatcaattgtttaaaaaaaaaaaaacgaaaaaaaaaaaaaaaacacaaaaaacccagaaaacttttaagtgttgctggaatatcctgcctacagtgggcacctccttgaagaagctgatagcttttacacagtattagattgaaataatggacagaaacacattcttgtcaagaaagggggagagaagtctgtttgcaagtttcaaagcaaaaagcaaaagtgaaatgatttgaggatttctgttctaatggagatgattctctgattgttagaaatggcaaatattgatgattgtgtgctattgattggtgcaggatacttggtatacgagtaaatacttgagactcgtgtcacttgataaattttctttttggactaggtcgcacagttattaaaacaacttttaaccctcccccttcacacacatacatatcaggttgttttctagttaaaaacccaagtagctcagattctactttaatgtcagtgcagatttgcattgaatcatgccattatgttttttctcatttttatgctgttgggtcttagtttttaaattgatataaagaactcagcaatggttttattttctactcatacttagggtttaggaaacactaccactagttatcatttaatcaacttcaatggtctactgaaacaaaaatggtaacttttcattagtggattatttagagttatagtagttgtttccagaaaacacttcctcacaattgtacttcccaatcaaatcatgtgatcatacagttattcccatgaaaggcagaatgtttgtttcaaaattaatctagttttctgtacatttaaatttgagaaggtgacaactggctcttttccagtcttccttcatgtcagttttctgatagaccactattggcaaacagtatctgtcaactaccaaatgtgtaaaattttctgtatttcactttgtcttatttgtaaatagtgaactaaaacttttggcagatcagcaacatttgctgagcctgttttttaagctaatgtgtattcttactaatgttcctatcaagaatggatttgtaatatatgctgtctatttctaatgttcacattcatattttgaggttctatcttattttaatagagaacagacttctcaaaaaatcttcagaagcagcttattattgaaatatcgaaatattgaaataaacccggtggggttagattactcatctgtccaccaagtgggacatttgcatggactgggggcttaaaggacttagaagagacctgtaagtaaatcctgaaaatgagccaatccccacttgaatggttactggagtaaacccacctttaccaccccaattacagcacccgaggccgataaaccaacttggctctggttcatttttcttttcttcatttgtgatgctcagattcaaaatgtgtgttctacactgttacaggcttctcttttgtttgattaaagattttagtcctacttttgtatggacacattagaatattcagagaccaaaatagaagaatttgctgttagatatttttcagaagtcagcagatttgtggcaaatcatttatttgcctttttaaaaattcatttaagcagttcagagagtagactactcagaaaattatttcacgtaattgtctaagaggtcaatattttttaatgcatattgaatcaaataaagtgctctaaagaaattattatacaaattcctttgggttgtttttcttttcttaacaaggggtgggggtaaacaggaatatgattcaggctttctggttgtgtatttaaagagtattgattttattattactattgatttactttattcctggcttccttttcacttttctttcaatttttaaaaaataatttaagccgttgaaaatataccaaactgttgaaacattttactcaaattttaaattcctaaaaatgttttttaataagagggagaaaattatttaaaaatacttatgcctatgccaatttccctcttttttcacaaaatccatgatttcagtttgtaagtagacatatatctaagggcacatttttggaaagtgaggaatagcagcagtataacttcattttgtcaggcctttgagttctaatattttgtattgctcttcaaatggatccttttaaaaaaattgtagataatgagtcataaatagattctgccaactgaggggagaaacattttaagtaaatatttttcagtatttggggccttaaaaaataattgtgtttccttaaaattacatgttagatagagtttttaggtttttttggttttaagattggttaaagcaatttaaaagccacttttttgtcaacatttaatagcctccacttctgttaagataatgtatactgctgaggaattactattaatagctatcaacataccaccattaaattaaggtattcactttagattttttattaaagcttttttcttgcacactgatcgttgtgtttctaagctgattttttcagctctaatatacctatggttaaaaagtataaaaacttaaattgatatttagatatatgttttcctattagtttatgttttaaaaagacaaaattgtatctgtcagtccctgaaggcagtttgtttttatactctctcacatttgtatttgttttttaaatggcagtattttagaagatttggagaaaagtccacataataatgttttcttaaaagcttttaaagtttttgctgtacttcaatttacttcttccatcagaaaactaagaacaaagtgttgctcagtctgttccgctgacctaaatttgtgttttcagcacttggctcagccaattcactgagtgaaggaattgctttatgaggcaaagcatgtgaaagttctaaagtatggttagattgtaggtcgtgctctatatggaaacatcaaaccattactacagagaaatgataaggcattggatccactattgaaattattatttttggatcaacaagttggtactttctgacttctgtatcttaacataagggaattttaggtaatgctaagtcagttgtctcattttttgtgataagttttggaatttttagttaattgaaataaataatgcttttaaatagaagtaaaaggtttataagtgtgcaaattgtagatttatcaattacctcagcaggtatcctgccatgtaattattagtgattagtgttaataagataatagattcaggtcttccaactatgcccttggattgtggcctactgtatgttattaaatggtctcttactatccaaaatgggagtagatgctgtggccccgtctcccttggcttttacgtcccatatccacccccattcatgtacaacatgtgaaatataaaaatctcatttcttgtcaaaatcagcactgcttatttgcatactcagcatcggatcagtgagtagttttataaaaaatccacgcacccaactcccttagttaaaacagattcttaattcataccatgaattcttaatttctgtaccatctatgttaatgatctgctgaaggtgactcaagattttcaaggtgtaatacagtttgatcatgtaccggacctggatatttaattttttttccctcacagttaatctcctccttgataaagcaataacactgctttgagtctgttgcctaatagcatgtcagaatcctctcctggatggtgattttataggaaagtttgtatgcatatcacccagtctatcttttaaaaattaagaaatttaaatgtatgctggaagtaatgacactatattgtggcattttattttaaaaattggggaaagttgcatatttttttaaaagtaagtgtttgagtaaaaaaattgaaggtacttttttaaggaaaaaaatttatatgccacagtttacatagacatttcagattcaacacgtactcttgaatataatggtttcttttacttggtcaaaatgcatgtatagcatttctttcatcttagttccttgtgtttgcctatgtggtcctttatatattttttattgtatcgaagaaacaaaactatcttcaaaaataagttaatttggatatatttgtcatatcaaactacaaagtgtacaaagttaagtttagcccttttctagaaagtgatctttaaaattaaaaatgctcctcttttaaattcaccaaatttatgtgtgggaaggcaccaaaatgattttgtaagtgccactgcaatattccctttcaagtgtggcctaaatttcaatcttaaggatggaatgcatgtctgctccttgttctgaaaaatgtaggcatctactacattttaaaacacagtgaaacatatacataagcctataaaaaaagatttgtgcaatttgaaagcctgttaattttttatgtagacatacctacacacgaaagggttaaattcacagccttactagttccttgcttccagtatttcaattggtctcctcccctcattattattattactactagtactattatttttgcacatagttaactgcccttcaatatgattcttaaaaagtgctgtttctgtggtatcgtattctctaaataatcatatttaattttttaaaacaaggttgcagtttctaattgtttcgttcctgtgtttttgctggtgtgtaataaaagcaagttttttcttttcatggttatttaatacattagctgcctgtaaataattcttgttataatgctctggaatgtgttgtagaagttgtattagattagttttaaacccttgtttgaaagccacattgttttggttatttctattaaattagaaaattgaaaaagttttcaaatgaa

SEQ ID NO: 3 - protein, 1110 aa, Homo sapiens
MAENKGGGEAESGGGGSGSAPVTAGAAGPAAQEAEPPLTAVLVEEEEEEGGRAGAEGGAAGPDDGGVAAASSGSAQAASSPAASVGTGVAGGAVSTPAPAPASAPAPGPSAGPPPGPPASLLDTCAVCQQSLQSRREAEPKLLPCLHSFCLRCLPEPERQLSVPIPGGSNGDIQQVGVIRCPVCRQECRQIDLVDNYFVKDTSEAPSSSDEKSEQVCTSCEDNASAVGFCVECGEWLCKTCIEAHQRVKFTKDHLIRKKEDVSESVGASGQRPVFCPVHKQEQLKLFCETCDRLTCRDCQLLEHKEHRYQFLEEAFQNQKGAIENLLAKLLEKKNYVHFAATQVQNRIKEVNETNKRVEQEIKVAIFTLINEINKKGKSLLQQLENVTKERQMKLLQQQNDITGLSRQVKHVMNFTNWAIASGSSTALLYSKRLITFQLRHILKARCDPVPAANGAIRFHCDPTFWAKNVVNLGNLVIESKPAPGYTPNVVVGQVPPGTNHISKTPGQINLAQLRLQHMQQQVYAQKHQQLQQMRMQQPPAPVPTTTTTTQQHPRQAAPQMLQQQPPRLISVQTMQRGNMNCGAFQAHQMRLAQNAARIPGIPRHSGPQYSMMQPHLQRQHSNPGHAGPFPVVSVHNTTINPTSPTTATMANANRGPTSPSVTAIELIPSVTNPENLPSLPDIPPIQLEDAGSSSLDNLLSRYISGSHLPPQPTSTMNPSPGPSALSPGSSGLSNSHTPVRPPSTSSTGSRGSCGSSGRTAEKTSLSFKSDQVKVKQEPGTEDEICSFSGGVKQEKTEDGRRSACMLSSPESSLTPPLSTNLHLESELDALASLENHVKIEPADMNESCKQSGLSSLVNGKSPIRSLMHRSARIGGDGNNKDDDPNEDWCAVCQNGGDLLCCEKCPKVFHLTCHVPTLLSFPSGDWICTFCRDIGKPEVEYDCDNLQHSKKGKTAQGLSPVDQRKCERLLLYLYCHELSIEFQEPVPASIPNYYKIIKKPMDLSTVKKKLQKKHSQHYQIPDDFVADVRLIFKNCERFNEADSEVAQAGKAVALYFEDKLTEIYSDRTFAPLPEFEQEEDDGEVTEDSDEDFIQPRRKRLKSDERPVHIK

SEQ ID NO: 4 - DNA, 8288 bp, Homo sapiens
ctgcggctggggctgggggcggcggcggcggcgacgcgggcggcgggcggcgcggggcggtccggcgggttcaaagaggaaaacatggcggaaaacaaaggcggcggcgaggctgagagcggcggcgggggcagcggcagcgcgccggtaactgccggggccgccgggcccgccgcgcaggaggcggagccgcctctcaccgcggtgctggtggaggaggaggaggaggaaggcggcagggccggcgctgagggcggcgcggccgggcccgacgacgggggggtggccgcggcctcctcgggctcggcccaggctgcttcatctcctgcggcctcagtgggcactggagttgccgggggcgcagtatcgacgccggctccagctccagcctcggctcccgctccgggtccctcggcagggccgcctcctggaccgccagcctcgctcctggacacctgcgccgtgtgtcagcagagcttgcagagccggcgtgaggcggagcccaagctgctgccctgtcttcactccttctgcctgcgctgcctgcccgagccggagcgccagctcagcgtgcccatcccggggggcagcaacggcgacatccagcaagttggtgtaatacggtgcccagtatgccgccaagaatgcagacagatagaccttgtggataattattttgtgaaagacacatctgaagctcctagcagttctgatgaaaaatcagaacaggtatgtactagttgtgaagacaatgcaagtgcagttggcttttgtgtagaatgtggagagtggctatgtaagacatgtatcgaagcacatcaaagagtaaaatttactaaagatcacttgatcaggaagaaagaagatgtctcagagtctgttggagcatctggtcaacgccctgttttctgccctgtacacaaacaagaacagttgaaacttttctgtgaaacatgtgatagattgacatgtagagactgtcagctattggaacacaaagaacataggtatcagtttttggaagaagcttttcaaaatcagaagggtgcaattgagaatctactggcgaaacttcttgagaagaagaattatgttcattttgcagctactcaggtgcagaataggataaaagaagtaaatgagactaacaaacgagtagaacaggaaattaaagtggccattttcacccttatcaatgaaattaataagaaaggaaaatctctcttacaacagctagagaatgttacaaaggaaagacagatgaagttactacagcagcagaatgacatcacaggcctttcccggcaggtgaagcatgttatgaacttcacaaattgggcaattgcaagtggcagcagcacagcactactatacagcaagcgactgattactttccagttgcgtcatattttgaaagcacggtgtgatcctgtccctgctgctaatggagcaatacgtttccattgtgatcccaccttctgggcaaagaatgtagtcaatttaggtaatctagtaatagagagtaaaccagctcctggttatactcctaatgttgtagttgggcaagttcctccagggacaaaccacattagtaaaacccctggacagattaacttagcacagcttcgactccagcacatgcaacaacaagtatatgcacagaaacatcagcagttgcaacagatgaggatgcagcaaccaccagcacctgtaccaactacaacaacaacaacacaacagcatcctagacaagcagcccctcagatgttacaacaacagcctcctcgattgatcagtgtgcaaacaatgcaaagaggcaacatgaactgtggagcttttcaagcccatcagatgagactggctcagaatgctgccagaataccagggatacccaggcacagcggccctcaatattccatgatgcagccacacctccaaagacaacactcaaacccagggcatgctggaccctttcccgtagtatcggtacacaacaccacaatcaacccaacgagccctactacagcaactatggcaaatgcaaaccgaggtcccaccagcccatctgttacagcaatagagctaatcccctcagttaccaatccagaaaaccttccatcgctgccagatattccacccatacagttggaagatgctggctcaagtagtttagataatctactaagtagatacatctcaggcagtcacctacccccacagcctacaagcaccatgaatccttctccaggtccctctgccctttctccgggatcatcaggtttatccaattctcacacacctgtgagacccccaagtacttctagtactggcagtcgaggcagctgtgggtcatcaggaagaactgctgagaagacaagtcttagtttcaaatctgatcaggtgaaggtcaagcaagaacctgggactgaagatgaaatatgtagcttttcaggaggtgtaaaacaagaaaaaacagaggatggcaggaggagtgcttgcatgttgagcagtcctgagagtagcttgacaccacctctctcaaccaacctgcatctagaaagtgaattggatgcattggcaagcctggaaaaccatgtgaaaattgaacctgcagatatgaatgaaagctgcaaacagtcagggctcagcagccttgttaatggaaagtccccaattcgaagcctcatgcacaggtcggcaaggattggaggagatggcaacaataaagatgatgacccaaatgaagactggtgtgctgtctgccaaaacggaggagatctcttgtgctgcgaaaaatgtccaaaggtctttcatctaacttgtcatgttccaacactacttagctttccaagtggggactggatatgcacattttgtagagatattggaaagccagaagttgaatatgattgtgataatttgcaacatagtaagaaggggaaaactgcgcaggggttaagccccgtggaccaaaggaaatgtgaacgtcttctgctttacctctattgccatgaattaagtattgaattccaggagcctgttcctgcttcgataccaaactactataaaattataaagaaaccaatggatttatccaccgtgaaaaagaagcttcagaaaaaacattcccaacactaccaaatcccggatgactttgtggccgatgtccgtttgatcttcaagaactgtgaaaggtttaatgaagctgattcagaagtagctcaggcagggaaagcagttgcattgtactttgaagataaactcacagagatctactcagacaggaccttcgcacctttgccagagtttgagcaggaagaggatgatggtgaggtaactgaggactctgatgaagactttatacagccccgcagaaaacgcctaaagtcagatgagagaccagtacatataaagtaaaatgacatggatttaaatcaattgtttaaaaaaaaaaaaacgaaaaaaaaaaaaaaaacacaaaaaacccagaaaacttttaagtgttgctggaatatcctgcctacagtgggcacctccttgaagaagctgatagcttttacacagtattagattgaaataatggacagaaacacattcttgtcaagaaagggggagagaagtctgtttgcaagtttcaaagcaaaaagcaaaagtgaaatgatttgaggatttctgttctaatggagatgattctctgattgttagaaatggcaaatattgatgattgtgtgctattgattggtgcaggatacttggtatacgagtaaatacttgagactcgtgtcacttgataaattttctttttggactaggtcgcacagttattaaaacaacttttaaccctcccccttcacacacatacatatcaggttgttttctagttaaaaacccaagtagctcagattctactttaatgtcagtgcagatttgcattgaatcatgccattatgttttttctcatttttatgctgttgggtcttagtttttaaattgatataaagaactcagcaatggttttattttctactcatacttagggtttaggaaacactaccactagttatcatttaatcaacttcaatggtctactgaaacaaaaatggtaacttttcattagtggattatttagagttatagtagttgtttccagaaaacacttcctcacaattgtacttcccaatcaaatcatgtgatcatacagttattcccatgaaaggcagaatgtttgtttcaaaattaatctagttttctgtacatttaaatttgagaaggtgacaactggctcttttccagtcttccttcatgtcagttttctgatagaccactattggcaaacagtatctgtcaactaccaaatgtgtaaaattttctgtatttcactttgtcttatttgtaaatagtgaactaaaacttttggcagatcagcaacatttgctgagcctgttttttaagctaatgtgtattcttactaatgttcctatcaagaatggatttgtaatatatgctgtctatttctaatgttcacattcatattttgaggttctatcttattttaatagagaacagacttctcaaaaaatcttcagaagcagcttattattgaaatatcgaaatattgaaataaacccggtggggttagattactcatctgtccaccaagtgggacatttgcatggactgggggcttaaaggacttagaagagacctgtaagtaaatcctgaaaatgagccaatccccacttgaatggttactggagtaaacccacctttaccaccccaattacagcacccgaggccgataaaccaacttggctctggttcatttttcttttcttcatttgtgatgctcagattcaaaatgtgtgttctacactgttacaggcttctcttttgtttgattaaagattttagtcctacttttgtatggacacattagaatattcagagaccaaaatagaagaatttgctgttagatatttttcagaagtcagcagatttgtggcaaatcatttatttgcctttttaaaaattcatttaagcagttcagagagtagactactcagaaaattatttcacgtaattgtctaagaggtcaatattttttaatgcatattgaatcaaataaagtgctctaaagaaattattatacaaattcctttgggttgtttttcttttcttaacaaggggtgggggtaaacaggaatatgattcaggctttctggttgtgtatttaaagagtattgattttattattactattgatttactttattcctggcttccttttcacttttctttcaatttttaaaaaataatttaagccgttgaaaatataccaaactgttgaaacattttactcaaattttaaattcctaaaaatgttttttaataagagggagaaaattatttaaaaatacttatgcctatgccaatttccctcttttttcacaaaatccatgatttcagtttgtaagtagacatatatctaagggcacatttttggaaagtgaggaatagcagcagtataacttcattttgtcaggcctttgagttctaatattttgtattgctcttcaaatggatccttttaaaaaaattgtagataatgagtcataaatagattctgccaactgaggggagaaacattttaagtaaatatttttcagtatttggggccttaaaaaataattgtgtttccttaaaattacatgttagatagagtttttaggtttttttggttttaagattggttaaagcaatttaaaagccacttttttgtcaacatttaatagcctccacttctgttaagataatgtatactgctgaggaattactattaatagctatcaacataccaccattaaattaaggtattcactttagattttttattaaagcttttttcttgcacactgatcgttgtgtttctaagctgattttttcagctctaatatacctatggttaaaaagtataaaaacttaaattgatatttagatatatgttttcctattagtttatgttttaaaaagacaaaattgtatctgtcagtccctgaaggcagtttgtttttatactctctcacatttgtatttgttttttaaatggcagtattttagaagatttggagaaaagtccacataataatgttttcttaaaagcttttaaagtttttgctgtacttcaatttacttcttccatcagaaaactaagaacaaagtgttgctcagtctgttccgctgacctaaatttgtgttttcagcacttggctcagccaattcactgagtgaaggaattgctttatgaggcaaagcatgtgaaagttctaaagtatggttagattgtaggtcgtgctctatatggaaacatcaaaccattactacagagaaatgataaggcattggatccactattgaaattattatttttggatcaacaagttggtactttctgacttctgtatcttaacataagggaattttaggtaatgctaagtcagttgtctcattttttgtgataagttttggaatttttagttaattgaaataaataatgcttttaaatagaagtaaaaggtttataagtgtgcaaattgtagatttatcaattacctcagcaggtatcctgccatgtaattattagtgattagtgttaataagataatagattcaggtcttccaactatgcccttggattgtggcctactgtatgttattaaatggtctcttactatccaaaatgggagtagatgctgtggccccgtctcccttggcttttacgtcccatatccacccccattcatgtacaacatgtgaaatataaaaatctcatttcttgtcaaaatcagcactgcttatttgcatactcagcatcggatcagtgagtagttttataaaaaatccacgcacccaactcccttagttaaaacagattcttaattcataccatgaattcttaatttctgtaccatctatgttaatgatctgctgaaggtgactcaagattttcaaggtgtaatacagtttgatcatgtaccggacctggatatttaattttttttccctcacagttaatctcctccttgataaagcaataacactgctttgagtctgttgcctaatagcatgtcagaatcctctcctggatggtgattttataggaaagtttgtatgcatatcacccagtctatcttttaaaaattaagaaatttaaatgtatgctggaagtaatgacactatattgtggcattttattttaaaaattggggaaagttgcatatttttttaaaagtaagtgtttgagtaaaaaaattgaaggtacttttttaaggaaaaaaatttatatgccacagtttacatagacatttcagattcaacacgtactcttgaatataatggtttcttttacttggtcaaaatgcatgtatagcatttctttcatcttagttccttgtgtttgcctatgtggtcctttatatattttttattgtatcgaagaaacaaaactatcttcaaaaataagttaatttggatatatttgtcatatcaaactacaaagtgtacaaagttaagtttagcccttttctagaaagtgatctttaaaattaaaaatgctcctcttttaaattcaccaaatttatgtgtgggaaggcaccaaaatgattttgtaagtgccactgcaatattccctttcaagtgtggcctaaatttcaatcttaaggatggaatgcatgtctgctccttgttctgaaaaatgtaggcatctactacattttaaaacacagtgaaacatatacataagcctataaaaaaagatttgtgcaatttgaaagcctgttaattttttatgtagacatacctacacacgaaagggttaaattcacagccttactagttccttgcttccagtatttcaattggtctcctcccctcattattattattactactagtactattatttttgcacatagttaactgcccttcaatatgattcttaaaaagtgctgtttctgtggtatcgtattctctaaataatcatatttaattttttaaaacaaggttgcagtttctaattgtttcgttcctgtgtttttgctggtgtgtaataaaagcaagttttttcttttcatggttatttaatacattagctgcctgtaaataattcttgttataatgctctggaatgtgttgtagaagttgtattagattagttttaaacccttgtttgaaagccacattgttttggttatttctattaaattagaaaattgaaaaagttttcaaatgaa

SEQ ID NO: 5 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 6 - DNA, 17 bp, Artificial Sequence
  PCR Primer hTIF1g-S
agcaacggcgacatcca

SEQ ID NO: 7 - DNA, 18 bp, Artificial Sequence
  PCR Primer hTIF1g-AS
tgcattcttggcggcata